SEQUENCE LISTING (7 sequences)

SEQ ID NO: 1 - DNA, 1301 bp, Homo sapiens
  misc_feature: Incyte ID No 238040
agagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgtgatggggtgcagacccccctcatggagtatggggcgctggacacatggccggggctgcaccagggacccatgggggctgcccagctggacagatggcttcctgctccccaggcccagccagggtcctctctcaaccactagacttggctctcaggaactctgcttcctggcccagcgctcgtgaccaaggatacaccaaagcccttaagacctcagggggcgggtgctggggtcttctccaataaatggggtgtcaaccgtttaaaaaaaaaaaaaagggcg

SEQ ID NO: 2 - DNA, 5679 bp, Homo sapiens
  misc_feature: Incyte ID No 411448
agcccccaaccccccgaggccactcacctcccccaactacccaggacagaggatgcccagccagccgagctccgggcagtacccgccccccacggtcaacatggggcagtattacaagccagaacagtttaatggacaaaataacacgttctcgggaagcagctacaagtaactacagccaagggaatgtcaacaggcctcccaggccggttcctgtggcaaattacccccactcacctgttccagggaaccccacaccccccatgacccctgggagcagcatccctccatacctgtcccccagccaagacgtcaaaccacccttcccgcctgacatcaagccaaatatgagcgctctgccaccacccccagccaaccacaatgacgagctgcggctcacattccctgtgcgggatggcgtggtgctggagcccttccgcctggagcacaacctggcggtcagcaaccatgtgttccacctgcggcccacggtccaccagacgctgatgtggaggtctgacctggagctgcagttcaagtgctaccaccacgaggaccggcagatgaacaccaactggcccgcctcggtgcaggtcagcgtgaacgccacgcccctcaccattgagcgcggcgacaacaagacctcccacaagcccctgcacctgaagcacgtgtgccagccgggccgcaacaccatccagatcaccgtcacggcctgctgctgctcccacctcttcgtgctgcagctggtacaccggccctccgtccgctctgtgctgcaaggactcctcaagaagcgcctcctgcccgcagagcactgtatcacgaaaatcaagcggaatttcagcagcgtggctgcctcctcgggcaacacgaccctcaacggggaggatggggtggagcagacggccatcaaggtgtctctgaagtgccccatcacattccggcgcatccagctgcctgctcgaggacacgattgcaagcatgtgcagtgctttgatctggagtcatacctgcagctgaattgcgagagagggacctggaggtgtcctgtgtgcaataaaaccgctctgctggagggcctggaggtggatcagtacatgtggggaatcctgaatgccatccaacactccgagtttgaagaggtcaccatcgatcccacgtgcagctggcggccggtgcccatcaagtcggacttacacatcaaggatgaccctgatggcatcccctccaagcggttcaagaccatgagtcccagccagatgatcatgcccaatgtcatggagatgatcgcagccctgggccccggcccgtccccctatcccctcccgcctcccccagggggcaccaactccaacgactacagcagccaaggcaacaactaccaaggccatggcaactttgacttcccccacgggaaccctggagggacatccatgaatgacttcatgcacgggcccccccagctctcccaccccccggacatgcccaacaacatggccgccctcgagaaacccctcagccaccccatgcaggaaactatgccacacgctggcagctctgaccagccccacccctccatacaacaaggtttgcacgtaccacaccccagcagccagtcagggcctccattacatcacagtggggctcctcctcnncatcnttcncagcntcnccggcagccgccacaggccgctcccagcagccatccacacagcgacctgacctttaacccctcctcagccttagagggtcaggccggagcgcagggagcgtccgacatgccggagccttcgctggatctccttcccgaactcacaaatcctgacgagctcctgtcttatctggacccccccgacctgccgagcaatagtaacgatgacctcctgtctctatttgagaacaactgagggccacccggtcgggcccatccctccacactctgcatcctaccccacctacccaacacacttttccacctgggagcctgtgccctcagaccgccccgcaccagagccacgggctgtggggcggggagccctcccccgctgcagccctctcagaacagaggggtagggagggtgcaccagtgcaccaggaaggctgtgtgggtctggagcccacgtcccacctccacacccttggcttgggcccatgcccagcgcaggcctgaagaccaccctcccgagaggaaccagcccggtaagagggcacacgctgatgcggcttcccggtccctccgcgtgtgccgattccaaatgaccttccagtgtccccaaggttcttccatcttctagactgtaaccctgcctccctgcttcctggtccagagcctccctccagtgactgtggagcctgagaaggcccccgggccccagcatgggccccgagccttggaggagcactggcagttggtggcagtgagaccaggccacccaccaccacccaccacagaaaagcacaaacctctgggaaagacaacgtctctcgggggccaggggtcatcggtttgacccctgacctataagccaagataccccataaacacactcagaaagcagagaaaaaggacaagagtctgtgtttgagagggggtctgccattcctgcttggggactggtggggaagagggccagacatcttctgagccagacgtccctgaggtccacctccaagctcagacaggcccaggctttgggaacagagagagcaggtgaacacccaaccaaagtgattgtgcccttggttggggggcgcgggcatataacctgtcagaagcaaacaggagcggcaacttctaactttgctccaagccactctctttttaaacagcaacaatttaaagctatgaagtcacctggagaaaaggaacgttgctcttggacagcaagcaaaccatttctctccgtctgttgtgtttttctcctagtacctcacctgccacctctccaagacttccgtgggacacccacttccctctgtcctagttctctttgtccaatcagatggcaagggcagtgcgtggaaaggccggggaggtgcagaaaccagagcccagggcaatggtgtctgtccagcccctccctctgtccctgtgctccaagcctgtcccccggctgcagcccaggccatggacatgtgcaccagtatgtacctgcaggcatcagggggaggggggcgtgtttctgggcctgccccagactactgcccttggctgccagcctaccctgcctgcactcctccaccatcacaatctcacccaaactcctgctcactcaagcaaaagcagcctctggccttccctccaccgctttgctccatctggcttaccactctccagggcctcctggggagcctgtcctgtgttcactttgtttcaggctggtctgtgccccgtgagccacatggcctagggtgatgccaggttgtcccgtcactggggtcccatctgtaaattctttgcgcccttcccggctgctgcctggggccctttcctgctctcccgtccgctgtgggtggtccccagcactcctctgtgggttttaccggaaaggtggccccagctgttgacttccagtcactgtcccagacggcacaaggttttctgtaggaaagctgccattgccccggccccttttcttcctttgtcccgttgtcgaggttttttcaaatagcgtgttgttcagtatgcaaatcaattattttaagaatcgcttttgtaaatatctttgtgaatattttagtatcgtctttgataatattcaacattttcatgacctggttatagcctttgctggtgtttttaaaatacctggactcaatgacaaagaccgagtcttctttgtgttctttaaacaaaaacaaaaaaagcaaccagggctatttgtacagttgaaggggtgaacagaatgggcggctgtgctgggagttggaagaccgggcagcccgctatttagagccatccctcagtcagctggcagggacaagccaacgccaggtagcatgtggccacccttgcccagtgtctgtggcctggcaagtggccacgccctgtgtcagaccatctgggaattaagctccagacagacttacagatgccttccttaggagttcttgcttcttgcgttgatactttgccccagaaaggcctgggattcattctggttcttatcagggtgtgtccacactctgctcacaggtggatccacggctttccagtgcagagagtcgagatgctccctgcagcccaggccccgggcacctcctgcaaccatctctgggctcagcacctgaggcgggtttcctgggtcccctctccagcaagcctccaccagcaagctcggcccagagcttcccttccggctggctctgaaccgtgcgtggtgcctacagcctgcagtctggagacaagctcttccggagtgctctgggagccaggccagggtgtgagggaggtgcagaggcatccggggcgggagcaagccccaggttgtgacaggtgcaggtagacaacgcccataaacagagatggtcctgaactctggagagatccttccctgatcctttcggacgactacttggagccataagtaacctcagcaaaaacgaggcctctgcaagccacttttccatgccaagcatccacccggcccacaggcatgtttctgccgccactccgcaagatggacagggagccagcaggcaggcgggaagggccaagtacaggcaatcacccccatcttcttggtttgaagctttatccatgtatcatgttccgtgtagccattttattttttaagaaactgctaatactttctccctaatggaagccctgatcccccagagagctacaggtctgctcccgacgggcctcgggcctgacccgtccacacagggccgtgtcaacagcagcgactcaagggacgtgtgtacatatgtaaatgagaaatagagacgtgtcaacagatgcattcatttctcttggaatgtgtattgtttttattttgcgaaacaaaacaaaacaaaaaaaaaagcttggaactccatcacgtggaaaaactagatcctgttggttatagcatttgtgagttctccacgtctgtctctctcgctcatgtaatatactctgaccctgagtggaaaggggtttttgttctgtttttattttacctacatgtactatttagcttcagtgtactagtcctgccacctgtgtatttttagggtgctatggaaataatgaaaagaaacggggatttcagaagaaaattgtaaccaaattcatactttgtataatttttgatatcatgatcacaggtgattcacacgtacacacataaacacacccaccagtgcagcctgaagtaactcccacagaaaccatcatcgtctttgtacatcgtatgtacaatgcaatcatttcatactttaaactggtcaaaaaactaattgtgatttctagtcttgcaaagctgtatgtagttagatgatgtgacaacctctaatatttatctaataaatatgtattcagatgaaacctgtatattaggtgttcatgtggttattttgtatttaaagatcaaattatttgactattgctagacatttctatactctgttgtaacactgaggtatctcatttgcccatgttaatttttttctaaataaattgacaaaaac

SEQ ID NO: 3 - DNA, 3513 bp, Homo sapiens
  misc_feature: Incyte ID No 454163
cggggaggaaggagtgtgcagagtgtcaccattcaggtgtcctgggaaagatgaaagcacccccgccaagaagacagacattccctggcggctgaagcagatgctggatatcctggtgtatgaagagcagcagcaggcggccgcggggtgaggcagggccctgcctggagtacctgctgcagcacaagatcctggagactctctgcacgctgggcaaggccgagtaccccccaggcatgcggcagcaggtgttccagttcttcagcaaggttctggcgcaggtgcagcaccccctgctgcattacctcagcgtccacaggcctgtgcagaaactcctccgacttggtgggactgcttccggatccgttacagaaaaggaggaggtgcagttcaccaccgtcctctgctccaagatccagcaggacccagagctgctcgcctacatcctggaaggtaaaaagattgtaggtaggaagaaagcatgcggagaacccactgccctgcctaaggacacaaccagccacggggacaaggactgctcccacgatggtgctcctgccaggccccagctggacggggagtcctgtggggcccaggccttgaacagccacatgcctgctgagaccgaggagctggacggtgggaccacagagagcaacctgattacctccctgcttgggctgtgccagagcaagaagagtcgggtggccttgaaggcccaggagaacctgctgctcctggtgagcatggcctccccagcagctgccacctacctggtacagagcagcgcctgctgccctgcgatcgtccggcacctttgccagttgtaccggtccatgcctgtcttcctggaccccgcagacattgccaccttagagggcatcagctggaggttacccagtgccccgtctgatgaggcttccttccctggcaaggaggccttggctgccttcttgggctggtttgattactgcgaccacctcatcacagaggcacacacggtggttgcggacgccttggcgaaggctgtggctgagaacttcttcgtggagaccctgcagccccagctcctgcacgtgtccgagcagagcatcttgacctccaccgccctcctcacagccatgctgcgccagcttcgctcccctgcgctgctgcgggaggccgtggctttcctcctgggcacagaccggcagcctgaagcccccggggacaacccccacaccctgtatgctcatctcatcgggcattgtgaccacctctctgatgagatcagcatcaccacactccggctgtttgaggagctgctgcagaagccccacgaggggatcatccacagcctggtcctgcgcaaccttgagggccgcccttacgtggcctggggctcaccagagcctgagagctatgaggacaccctagacctggaggaagacccctacttcaccgacagcttcctggattccggctttcaaactcccgcaaagcctcgcctagctcctgctaccagttacgatggcaaaacagcagtgaccgagatcgtcaacaggagtgcagctcccgcgtcgcctcctggggctggcctctgacccccacacctttggacccccatgagcccgagcgacctttcttcgagggccacttcctccgagtgctgtttgaccgcatgtcccggattctggatcagccatacagcctgaacctgcaggtgacctcggtcctgtcccggcttgccctcttcccccacccccatattcatgagtacctgctggatccgtacatcagcctggcccccggctgcaggagcctattctccgtgttggtgagggtgatcggggacttgatgcagagaatccagagggtaccccagttcccaggcaagctgctcctggtgcgcaacagttgacgggccaggctcctggggagcagctggaccaccagaccctcctccagggcgtggtggtgctggaggagttctgcaaggagctggctgccattgccttcgtcaagtttcccccacatgatcctcgccagaacgtctccccagccccggaagggcaggtctgagccagcaccagggcggtgggagactcctgtccacacctctgccccagagctgcctcctgcctggcactgccgccacactcccctcctgggatggggcttctgctcccgggctcactcaaggagactgcggcatgttgaccacaccagactgggtttcagggaatgggcatgccaggtgccaaggagccaaacagatggctttccaggcagcaaggtccttggggccttcttggaggagcttgggtgacagccaggtgagcacccagaccccagaccctcatgtgctgtgtgcctggccccttctgtactggccatttgtggccagggccaagcctgtgactcaactccaggggcaagatggggagtgagctgatggctccgagactggtcaggagcccaggccagtgagatggggcctggagccttgtctgtgtcacattaggtaccatgggagctgctgagacctgacattttgtcccctgcctacatggcttggcccatggagaaggagcagtgaatgggatcgtcggggaagcccctcttcctgctctgctcccctggaaactgttgcaaaactcccagccgcctcatggcaaatgcccaaagcatgttccgcacccaggcgggggcccctgctaatgagaaccttggtgcagctgcagccaggaggggagcgggcccaggagccaggctcaggtccagctggttcctctctggcgccttctgaacccgtctcagcaggtccacagcacctgggcagaggtcagagaccaggggaggccgggccttgccctcccttctgcccagggcccagtgttcttgatagaagacccttctggggagccagggagctcaggggacagataagggaaggacgccccctgactccaggcccctgagcctggcgggaagtggctgcggcccaggcagccagtcctggtggtgttctccctgcatgccctccgtggctgggctgccaccccacccggcccgaatctgtcttgacctgcaggaatacacgggcggcgccaggcattacctcacagcgggactacacagttgctggctttgctcctgggcaaggaggagcaggccagagcctcttttgcttccttttcttgcccatgccgcttctagaagccaggcacaggttgccaagaggtgacacgaaacaggaggaaactcagtgacctctgcctctcccacattcctccccgcgggggaggacctcgccgctctgaagagcaccgtgcacatgtgggtgcacaaacgtgggtgttggtgtggacggggcgcagatctccgtggatgaactgcgtctggactcttagattcataaaatattcgagggtttgggagtcacagaccctcccctctcctcagtgcactttggcatttgcacggtgtcttccccggacagcacagcaataaatggtgtgattgcgtggaaaa

SEQ ID NO: 4 - DNA, 2970 bp, Homo sapiens
  misc_feature: Incyte ID No 988966.17
gtgggtacaagatgacggagccgggcgcctctcccgaggacccttgggtcaaggcaagccccgtgggcgcgcacgccggcgaggggagggcgggtcgggctcgtgcacgtaggggggccggaagacgaggggcttccctcctgtccccaaagtcccccacgctctccgtgccccggggctgcagagaagacagctctcaccccgcgtgtgccaaggtggagtatgcctacagcgacaacagcctggaccccgatgatgaggacagtgattaccaccaggaggcctacaaggagtcctacaaagaccggcggcggcgcgcacacactcaggctgagcagaagaggagggacgccatcaagagaggctatgatgaccttcagaccatcgtccccacttgccagcagcaggacttctccattggctcccaaaagctcagcaaagccatcgttctacaaaagaccattgactacattcagtttttgcacaaggagaagaaaaagcaggaggaggaggtgtccacgttacgcaaggatgtcaccgccctaaagatcatgaaagtgaactatgagcagattgtgaaggcacaccaggacaacccccatgaaggggaggaccaggtctctgaccaggtcaagttcaacgtgtttcaaggcatcatggattccctgttccagtccttcaatgcctccatctcagtggccagcttccaggagctgtcagcatgtgtcttcagctggatcgaggagcactgtaagcctcagaccctgcgggagattgtgattggcgtcctgcaccaattgaaaaaccagctttactgaccggttcttggaaacctggagaacagccaacaagaggcccttgaatctctacgtggccactgaactgctgggcccgggagactggactacaacacctcacactggtcagctggtttctacttggtgtttggtttttcccagccccattttatcttcagcggagccgcggtgtttgttttgtgaaagcttctgattaatttattatattgacgataaaactcaaacctacccagccttccccccactccatggaagtccttgggatgggcgtctgctctggacaccccaaagagctcctgccctctcagccctttattcaagcctcagatttctgctcatgatctacatagatttggaaactgttttcctctgttttggtctcttgggcaacatttttggcccaagtttgggcaacatttggcccaagtttgggcattttggcagtagctgtatgggagaaaaagagtaagaggaaatattcccacagccatgaagggtgaaagggcaccttgtgcctagactagggctgcctggtcagtcccaggtgaggccaagggctttctggccatctcagggaggggccaccaggttcctcccctcaccccatattccatcaccttcctcctctgctctgggtggtaagggaagccctcccggttcccacaggctatgatgctgcatggcagaggcaggtataacacagcactacatattggaaattttttatttttctaaataccaatgcagttttgctacggttacaattttgaaatattaactgagcctcaaaatcaccctttctgtcaagcatatcttggcctctcccatgtctcagtgttgcctgcatttctcccaggacttgggggtggggtgaaaagcgtacaaaagatacttaaaagggctcctggggtacacaagcccagcaggtcctgagtgaagccgtgggccctccaaatgctcgttttatagcaacctctctctaccctagttctccaaattcacttctgccttcctcaggtttgatatctggcaggtttgactatccagaggaaattaaatatttttatataaaattaaattataataaatattgccaaatgctttcctttagcattgttccaagtctaaatgttaacctcaagctactgcaatttagacaatgaaatgggctgggtctacccccagccaccagccctcatcctctctacccagtgctctggtttatgcttgtctcctgactgctctgcttaaaggtgaaagtagcaggaacaacaacaaaagccaaccaaaaacaaggtagccagtgcaagacatctcactcttctgacatcctgcagtccccaccagtcctgaccgtgggcccctcaggggtctgggagtgtgacgttgtaatcttcatccgtctctatcccaacttcctcaaagaactgcttcttgctcttggggtatccttcaagtattgcatcagacagctctgtagccatcctcttcctcttcctccactccttacagtacttctgcctctctctgtacacctgctctttctcttctggagtcacatgattggtagctgctttaatgttcttcaatctctctctgtagccagcggcattccttctttaactcctggatttctttctgcatctctggtgtggtcagggcactagataattccttgagctcagcctccatgtagcggcagctctgctgcaagctctgcaccttagcagtgagggccacgattttgccatctaggacttgaaggtcagcatcactcaccatgtcaaactggtcctgatccgcaaaatagatcttctgcttgccgtacatcttctctttgatcttgccttgttgcgccagctgctccagcgtcttcaccaccaccgccttgcccagtccgtgttcccgctgtaggttcccgaacacatcctgggagctgtagggccggttctgctcctgcaggtacctcaggaggatcccggcggctcccgccgcagcttctgcccggcctttactcatcgcctttcccgccacccaactcagaaagccggacgttgtagttggtggtccgggcgcggtagcgggctcgtggaaaaccccggtctgttgtgctgatgcctggtctgttggttcgttttgggatcaccttgat

SEQ ID NO: 5 - DNA, 1671 bp, Homo sapiens
  misc_feature: Incyte ID No 254749.3
ctggtcgtcgtctgcngcggctgcggcggctgaggagcccggctgaggcgccagtacccggcccggtccgcatttccgccttccggcttcggtttccctcggcccagcacgccccggccccgccccagccctcctgatccctcgcagcccggctccggccgcccgcctctgccgccgcaatgatgatgatggcgctgagcaagaccttcgggcagaagcccgtgaagttccagctggaggacgacggcgagttctacatgatcggctccgaggtgggaaactacctccgtatgttccgaggttctctgtacaagagatacccctcactctggaggcgactagccactgtggaagagaggaagaaaatagttgcatcgtcacatggtaaaaaaacaaaacctaacactaaggatcacggatacacgactctagccaccagtgtgaccctgttaaaagcctcggaagtggaagagattctggatggcaacgatgagaagtacaaggctgtgtccatcagcacagagccccccacctacctcagggaacagaaggccaagaggaacagccagtgggtacccaccctgcccaacagctcccaccacttagatgccgtgccatgctccacaaccatcaacaggaaccgcatgggccgagacaagaagagaaccttccccctttgctttgatgaccatgacccagctgtgatccatgagaacgcatctcagcccgaggtgctggtccccatccggctggacatggagatcgatgggcagaagctgcgagacgccttcacctggaacatgaatgagaagttgatgacgcctgagatgttttcagaaatcctctgtgacgatctggatttgaacccgctgacgtttgtgccagccatcgcctctgccatcagacagcagatcgagtcctaccccacggacagcatcctggaggaccagtcagaccagcgcgtcatcatcaagctgaacatccatgtgggaaacatttccctggtggaccagtttgagtgggacatgtcagagaaggagaactcaccagagaagtttgccctgaagctgtgctcggagctggggttgggcggggagtttgtcaccaccatcgcatacagcatccggggacagctgagctggcatcagaagacctacgccttcagcgagaaccctctgcccacagtggagattgccatccggaacacgggcgatgcggaccagtggtgcccactgctggagactctgacagacgctgagatggagaagaagatccgcgaccaggacaggaacacgaggcggatgaggcgtcttgccaacacggccccggcctggtaaccagcccatcagcacacggctcccacggagcatctcagaagattgggccgcctctcctccatcttctggcaaggacagaggcgaggggacagcccagcgccatcctgaggatcgggtgggggtggagtgggggcttccaggtggcccttcccggcacacattccatttgttgagccccagtcctgccccccaccccaccctccctacccctccccagtctctggggtcaggaagaaaccttattttaggttgtgttttgtttttgtataggagccccaggcagggctagtaacagtttttaaataaaaggcaacaggtcatgttcaatttcttc

SEQ ID NO: 6 - protein, 635 aa, Homo sapiens
  misc_feature: Incyte ID No 411448
PPTPRGHSPPPTTQDRGCPASRAPGSTRPPRSTWGSITSQNSLMDKITRSREAATSNYSQGNVNRPPRPVPVANYPHSPVPGNPTPPMTPGSSIPPYLSPSQDVKPPFPPDIKPNMSALPPPPANHNDELRLTFPVRDGVVLEPFRLEHNLAVSNHVFHLRPTVHQTLMWRSDLELQFKCYHHEDRQMNTNWPASVQVSVNATPLTIERGDNKTSHKPLHLKHVCQPGRNTIQITVTACCCSHLFVLQLVHRPSVRSVLQGLLKKRLLPAEHCITKIKRNFSSVAASSGNTTLNGEDGVEQTAIKVSLKCPITFRRIQLPARGHDCKHVQCFDLESYLQLNCERGTWRCPVCNKTALLEGLEVDQYMWGILNAIQHSEFEEVTIDPTCSWRPVPIKSDLHIKDDPDGIPSKRFKTMSPSQMIMPNVMEMIAALGPGPSPYPLPPPPGGTNSNDYSSQGNNYQGHGNFDFPHGNPGGTSMNDFMHGPPQLSHPPDMPNNMAALEKPLSHPMQETMPHAGSSDQPHPSIQQGLHVPHPSSQSGPPLHHSGAPPXHXSQXXRQPPQAAPSSHPHSDLTFNPSSALEGQAGAQGASDMPEPSLDLLPELTNPDELLSYLDPPDLPSNSNDDLLSLFENN

SEQ ID NO: 7 - protein, 268 aa, Homo sapiens
  misc_feature: Incyte ID No 988966.17
MTEPGASPEDPWVKASPVGAHAGEGRAGRARARRGAGRRGASLLSPKSPTLSVPRGCREDSSHPACAKVEYAYSDNSLDPDDEDSDYHQEAYKESYKDRRRRAHTQAEQKRRDAIKRGYDDLQTIVPTCQQQDFSIGSQKLSKAIVLQKTIDYIQFLHKEKKKQEEEVSTLRKDVTALKIMKVNYEQIVKAHQDNPHEGEDQVSDQVKFNVFQGIMDSLFQSFNASISVASFQELSACVFSWIEEHCKPQTLREIVIGVLHQLKNQLY

(19) United States
(12) Patent Application Publication
(10) Pub. No.: US 2003/0044812 A1
(43) Pub. Date: Mar. 6, 2003

(54) Cell differentiation cDNAs induced by retinoic acid

(76) Inventors: Michael G. Walker, Sunnyvale, CA (US); Peter J. van der Spek, Papendrecht (NL); Andreas Kremer, Leverkusen (DE); Lynn E. Murry, Fayetteville, AR (US)

Correspondence Address: INCYTE GENOMICS, INC., 3160 PORTER DRIVE, PALO ALTO, CA, 94304, US

(21) Appl. No.: 10/053,812

(22) Filed: Jan. 18, 2002

Related U.S. Application Data
(60) Provisional application No. 60/263,031, filed on Jan. 18, 2001.

(51) Int. Cl. C07K 14/705; C12Q 1/68; C12P 21/02; C12N 5/06; C07H 21/04; G01N 33/543

(52) U.S. Cl. 435/6; 436/518; 530/350; 435/69.1; 435/320.1; 435/325; 514/12; 536/23.5

(57) ABSTRACT

The invention provides compositions and novel cDNAs that co-express with cell differentiation gene transcripts induced by retinoic acid. The invention also provides expression vectors, host cells, proteins encoded by the cDNAs and antibodies which specifically bind the proteins. The invention also provides methods for the diagnosis, prognosis and treatment of and disorders associated with cell and tissue development and differentiation.

This application claims the benefit of provisional application Serial No. 60/263,031, filed Jan. 18, 2001.

### FIELD OF THE INVENTION

The invention relates to cDNAs produced from transcripts induced by retinoic acid and to their use in diagnosis, prognosis, and treatment of cancer and disorders associated with cell differentiation.

### BACKGROUND OF THE INVENTION

Retinoic acid (RA) is the bioactive metabolite of vitamin A (retinol) which acts on cells to establish or change the pattern of gene activity. RA is well known for its importance in the regulation of cell growth and differentiation (Perez-Castro et al. (1989) Proc Natl Acad Sci 86: 8813-8817; Wei et al. (1989) Mol Endocrinol 3: 454-463), and in the prevention of cancer. In its post-embryonic role, RA has been shown to play a part in limb, spinal cord, lung, and hair cell regeneration. RA may act alone, in association with its receptors, or in combination with melatonin, lipid-soluble vitamins, cytokines and other growth factors, and extracellular matrix molecules such as fibronectin. As an anti-tumor agent, RA is known to induce differentiation or apoptosis (Hansen et al. (2000) Carcinogenesis 21: 1271-1279; Manna and Aggarwal (2000) Oncogene 19: 2110-2119; and Martinet et al. (2000) Cancer Res 60: 2869-2875).

Although several RA-related compounds have demonstrated anti-tumor effects and are in clinical trials for human therapy (De Coster et al. (1996) J Steroid Biochem Mol Biol 56: 133-143; Krekels et al. (1996) Prostate 29: 36-41; and Zhang et al. (2000) J Cell Physiol 185: 1-20), the genes responsible for these effects of RA have only partially been elucidated.

Identification of additional cDNAs whose transcripts are induced by RA and participate in the process of cell differentiation satisfies a need in the art by providing new compositions which are useful in the diagnosis, prognosis, and treatment of individuals with cancer or in need of cell or tissue-specific developmental intervention.

### SUMMARY OF THE INVENTION

The invention provides for a combination comprising a plurality of cDNAs having the nucleic acid sequences of SEQ ID NOs: 1-5 that are co-expressed with one or more known genes whose transcripts are induced by retinoic acid or the complements of SEQ ID NOs: 1-5. The invention also provides an isolated cDNA having a nucleic acid sequence selected from SEQ ID NOs: 1-5 and the complements thereof. In one aspect, the combination is used in the diagnosis, prognosis, and treatment of cancer and disorders associated with cell differentiation. In another aspect, the cDNA is used as a probe, in an expression vector, or in a composition in combination with a labeling moiety.

The invention provides a method for using a combination or a cDNA of the invention to screen a plurality of molecules to identify ligands, molecules or compounds, which bind the cDNA. The molecules or compounds are selected from DNA molecules, RNA molecules, peptide nucleic acids (PNAs), mimetics, and proteins. In one embodiment, the combination or the cDNA are attached to a substrate. In one aspect, the substrate is used to detect gene expression in a diagnosing a cancer or cell differentiation disorder. The method comprises hybridizing the substrate containing the cDNA to a sample under conditions for formation of one or more hybridization complexes, detecting hybridization complex formation; and comparing the amount of complex formation with the amount of complex formation in a non-diseased sample, wherein the altered amount of complex formation indicates the presence of the cancer or cell differentiation disorder.

The invention provides a purified protein encoded by a cDNA of the invention that is coexpressed with one or more known retinoic acid induced genes in a plurality of biological samples. The invention also provides a method for using a protein to screen a plurality of molecules to identify at least one ligand which specifically binds the protein. The molecules are selected from DNA molecules, RNA molecules, peptide nucleic acids, proteins, agonists, antagonists, and antibodies. The invention further provides a method of using a protein to purify a ligand.

The invention provides a method of using a protein to prepare and purify an antibody that specifically binds to the protein of the invention. The invention also provided a purified antibody. The invention further provides a composition comprising a cDNA, a protein or an antibody that specifically binds a protein and a pharmaceutical carrier.

The invention provides a method for using an antibody to detect expression in a sample, the method comprising combining the antibody with a sample under conditions which allow the formation of antibody:protein complexes; and detecting complex formation, wherein complex formation indicates expression of the protein in the sample. In one embodiment, complex formation is compared with standards and is diagnostic of a disorder associated with steroid-responsive tissues or pregnancy. The invention also provides a method for using an antibody to immunopurify a protein comprising attaching the antibody to a substrate; contacting the antibody with solution containing the protein, thereby forming an antibody:protein complex; dissociating the antibody:protein complex; and collecting the purified protein.

### BRIEF DESCRIPTION OF THE SEQUENCE LISTING

The Sequence Listing provides exemplary cDNAs comprising the nucleic acid sequences of SEQ ID NOs:1-5. Each sequence is identified by a sequence identification number (SEQ ID NO) and by the Incyte number with which the sequence was first identified.

### DESCRIPTION OF THE INVENTION

It must be noted that as used herein and in the appended claims, the singular forms “a”, “an”, and “the” include the plural reference unless the context clearly dictates otherwise. Thus, for example, a reference to “a host cell” includes a plurality of such host cells, and a reference to “an antibody” is a reference to one or more antibodies and equivalents thereof known to those skilled in the art.

It is to be understood that this invention is not limited to the particular devices, machines, materials and methods described. Although particular embodiments are presented, equivalent embodiments may be used to practice the invention. The embodiments are provided to illustrate the invention and are not intended to limit the scope of the invention which is limited only by the appended claims.

Definitions

“Array” refers to an ordered arrangement of at least two cDNAs, proteins, or antibodies on a substrate. At least one of the cDNAs, proteins, or antibodies represents a control or standard, and the other, a cDNA, protein, or antibody of diagnostic or therapeutic interest. The arrangement of two to about 40,000 cDNAs, proteins, or antibodies on the substrate assures that the size and signal intensity of each labeled complex, formed between each cDNA and at least one nucleic acid, or antibody:protein complex, formed between each antibody and at least one protein to which the antibody specifically binds, is individually distinguishable.

“Cancer” refers to any cancer including, but not limited to, an adenocarcinoma, leukemia,; lymphoma, melanoma, myeloma, sarcoma, and teratocarcinoma of the adrenal gland, bladder, blood, bone, bone marrow, brain, breast, gastrointestinal tract, heart, kidney, liver, lung, lymph, muscle, nerve, ovary, pancreas, prostate, skin, spleen, stomach, testis, and uterus and particularly cancers or tumors of the bladder, brain, breast, colon, endometrium, ovary, prostate, testicles and uterus.

“cDNA” refers to an isolated polynucleotide. It may be of genomic or synthetic origin, double-stranded or single-stranded, and combined with other cDNAs, vitamins, minerals, carbohydrates, lipids, proteins, or other types of nucleic acids to perform a particular activity or to form a useful composition.

A “combination” refers to at least two and up to 5 cDNAs have nucleic acid sequences selected from SEQ ID NOs: 1-5 and their complements as presented in the Sequence Listing.

The “complement” of a nucleic acid molecule of the Sequence Listing refers to a cDNA which is completely complementary over the full length of the sequence and which will hybridize to the nucleic acid molecule under conditions of high stringency.

“Differential expression” refers to an increased, upregulated or present, or decreased, downregulated or absent, gene expression as detected by the absence, presence, or at least two-fold changes in the amount of transcribed messenger RNA or translated protein in a sample.

“Disorders associated with cell differentiaion” refers to conditions, diseases and disorders such as amyotrophic lateral sclerosis, Alzheimer disease, amyloidosis, asthma, ataxias, cerebral agenesis, collagen vascular diseases, diabetes, ductus arteriosus, Huntington disease, hypoplastic left heart, psoriasis, retinal disease, rheumatoid arthritis, Smith-Magenis syndrome, and scleroderma.

“Isolated or purified” refers to a cDNA or protein that is removed from its natural environment and that is separated from other components with which it is naturally present.

“Labeling moiety” refers to any reporter molecule, visible or radioactive label, than can be attached to or incorporated into a cDNA, protein or antibody. Visible labels include but are not limited to anthocyanins, green fluorescent protein (GFP), β glucuronidase, luciferase, Cy3 and Cy5, and the like. Radioactive markers include radioactive forms of hydrogen, iodine, phosphorous, sulfur, and the like.

“Ligand” refers to any agent, molecule, or compound which will bind specifically to a complementary site on a cDNA molecule or polynucleotide, or to an epitope or a protein. Such ligands stabilize or modulate the activity of polynucleotides or proteins and may be composed of inorganic or organic substances including nucleic acids, proteins, carbohydrates, fats, and lipids.

A “portion” of a protein refers to that length of amino acid sequence which would retain at least one biological activity, a domain identified by PFAM or PRINTS analysis or an antigenic epitope of the protein identified using Kyte-Doolittle algorithms of the PROTEAN program (DNASTAR, Madison, Wis.).,

“Protein” refers to a polypeptide, or a portion thereof, whether naturally occurring, recombinant, or synthetic. An “oligopeptide” is an amino acid sequence from about five residues to about 15 residues that is used as part of a fusion protein to produce an antibody.

“Retinoic acid induced gene” refers to a polynucleotide which has been previously identified as useful in the diagnosis, prognosis, or treatment of cancer or disorders associated with cell differentiation. Typically, this means that the known gene is differentially expressed at higher (or lower) levels in tissues from patients with the disorder when compared with normal expression in any tissue. The “known retinoic acid induced genes” as identified by library subtraction methodology are: P450 retinoic acid induced 1 and 2 (RAI-½), retinoic acid hydroxlase (CYP26), laminin beta-2 chain (laminin β-2), retinol-binding-protein receptor p63 (RBPR-p63), P97, lamin A, beta-2-microglobulin (β-2-m), and amyloid precursor like protein 2 (APLP2). The known cell differentiation genes are collagens, notch 3, platelet derived growth factor (PDGF), fibulin-2, fibrillin, insulin growth factor-1 (IGF-I), cAMP-dependent protein kinase regulatory subunit RI beta (RPK-RI), SWAP, EMP, YPT3/rab11, ERK-1, and β4 integrin.

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a biopsy, a cell; a tissue; a tissue print; a fingerprint, buccal cells, skin, or hair; and the like.

“Specific binding” refers to a special and precise interaction between two molecules which is dependent upon their structure, particularly their molecular side groups. For example, the intercalation of a regulatory protein into the major groove of a DNA molecule, the hydrogen bonding along the backbone between two single stranded nucleic acids, or the binding between an epitope of a protein and an agonist, antagonist, or antibody.

“Substrate” refers to any rigid or semi-rigid support to which cDNAs or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “variant” refers to a cDNA or protein whose sequence diverges from about 5% to about 30% from the nucleic acid or amino acid sequences of the Sequence Listing.

### THE INVENTION

The present invention utilizes a method for identifying cDNAs and proteins that are associated with a signaling or regulatory pathway specific disease, subcellular compartment, cell type, tissue, or species. In particular, the method identifies cDNAs derived from transcripts of genes which are induced by retinoic acid and that are co-expressed with genes known to be involved in cell differentiation. These genes and cDNAs are useful in diagnosis, prognosis, treatment, and evaluation of therapies for cancers and disorders associate with cell and tissue development and differentiation.

Expression levels of genes in two cell lines, a bone marrow-derived cell line and a fibroblast derived cell line, before and after treatment with retinoic acid (RA) were compared using library subtraction. The bone-marrow cDNA libraries were produced from cells derived from a metastatic bone marrow neuroblastoma removed from a 4-year old Caucasian female. The samples were either untreated or treated with 10 μM RA and cultured for four days. The fibroblast libraries were produced from cells derived from fibroblast tissue removed from the breast of a 31-year old Caucasian female. The samples were either untreated or treated with 1 μM RA for 20 hours.

The genes and cDNAs identified by library subtraction methodology and expressed at high levels in the retinoic acid-treated libraries and not detected in the untreated libraries include:

| Gene/cDNA* | Known association with RA |
|---|---|
| RAI-1/2 | Yes |
| SEQ ID NO:1 (Incyte | No previous report; uncharacterized cDNA |
| 238040) | |
| SEQ ID NO:2 (Incyte | No previous report; uncharacterized cDNA |
| 411448) | |
| CYP26 | Yes |
| Laminin β-2 | Yes |
| RBPR-p63 | Yes |
| SEQ ID NO:5 (Incyte | No previous report; tumor suppressor |
| 254749) | |
| SEQ ID NO:3 (Incyte | No previous report; uncharacterized cDNA |
| 454163) | |
| P97 | Yes |
| SEQ ID NO:4 (Incyte | No previous report; uncharacterized cDNA |
| 988966) | |
| PB-0017 US | |
| Lamin A | Yes |
| β-2-m | Yes |
| APLP2 | Yes |

*These genes are ordered by expression level with the highest expression occurring in RAI-½.

The cDNAs shown above by SEQ ID NO and Incyte number were further analyzed using the guilt by association method (Walker and Volkmuth (1999) Prediction of gene function by genome-scale expression analysis: prostate cancer-associated genes. Genome Res 9: 1198-1203). The method provides for the identification of cDNAs that are expressed in a plurality of libraries. The cDNAs include genes of known or unknown function which are expressed in a specific signaling pathway, disease process, subcellular compartment, cell type, tissue, or species. The expression patterns of genes with known function are compared with those of cDNAs with unknown function to determine whether a specified co-expression probability threshold is met. Through this comparison, a subset of the cDNAs having a high co-expression probability with the known genes can be identified. The high co-expression probability correlates with a particular co-expression probability threshold which is preferably less than 0.001 and more preferably less than 0.00001.

The cDNAs originate from cDNA libraries derived from a variety of sources including, but not limited to, eukaryotes such as human, mouse, rat, dog, monkey, plant, and yeast; prokaryotes such as bacteria; and viruses. These cDNAs can also be selected from a variety of sequence types including, but not limited to, expressed sequence tags (ESTs), assembled polynucleotides, full length gene coding regions, promoters, introns, enhancers, 5′ untranslated regions, and 3′ untranslated regions. To have statistically significant analytical results, the cDNAs are expressed in at least five cDNA libraries.

The 1176 cDNA libraries used in the co-expression analysis of the present invention can be obtained from adrenal gland, biliary tract, bladder, blood cells, blood vessels, bone marrow, brain, bronchus, cartilage, chromaffin system, colon, connective tissue, cultured cells, embryonic stem cells, endocrine glands, epithelium, esophagus, fetus, ganglia, heart, hypothalamus, immune system, intestine, islets of Langerhans, kidney, larynx, liver, lung, lymph, muscles, neurons, ovary, pancreas, penis, peripheral nervous system, phagocytes, pituitary, placenta, pleura, prostate, salivary glands, seminal vesicles, skeleton, spleen, stomach, testis, thymus, tongue, ureter, uterus, and the like. The number of cDNA libraries selected can range from as few as 5 to greater than 10,000 and preferably, the number of the cDNA libraries is greater than 500.

In a preferred embodiment, the cDNAs are assembled from related sequences, such as sequence fragments derived from a single transcript. Assembly of the polynucleotide can be performed using sequences of various types including, but not limited to, ESTs, extension of the EST, shotgun sequences from a cloned insert, or full length cDNAs. In a most preferred embodiment, the cDNAs are derived from human sequences that have been assembled using the algorithm disclosed in U.S. Ser. No. 9,276,534, filed Mar. 25, 1999, incorporated herein by reference.

Experimentally, differential expression of the polynucleotides can be evaluated by methods including, but not limited to, differential display by spatial immobilization or by gel electrophoresis, genome mismatch scanning, representational difference analysis, and transcript imaging. Additionally, differential expression can be assessed by microarray technology. These methods may be used alone or in combination.

Known retinoic acid-induced genes can be selected based on the use of the genes as diagnostic or prognostic markers or as therapeutic targets for cancer or disorders associated with cell differentiation. Preferably, the cell differentiation genes induced by retinoic acid are RAI-1/2, CYP26, laminin β-2, RBPR-p63, P97, lamin A, β-2-m, APLP2, collagens, notch 3, PDGF, fibulin-2, fibrillin, IGF-I, RPK RI, SWAP, EMP, YPT3/rab11, ERK-1, and β4 integrin.

The procedure for identifying novel cDNAs that exhibit a statistically significant co-expression pattern with known retinoic acid induced genes is as follows. First, the presence or absence of a gene sequence in a cDNA library is defined: a gene is present in a cDNA library when at least one cDNA fragment corresponding to that gene is detected in a cDNA sample taken from the library, and a gene is absent from a library when no corresponding cDNA fragment is detected in the sample.

Second, the significance of gene co-expression is evaluated using a probability method to measure a due-to-chance probability of the co-expression. The probability method can be the Fisher exact test, the chi-squared test, or the kappa test. These tests and examples of their applications are well known in the art and can be found in standard statistics texts (Agresti (1990) Categorical Data Analysis, John Wiley & Sons, New York, N.Y.; Rice (1988) Mathematical Statistics and Data Analysis, Duxbury Press, Pacific Grove, Calif.). A Bonferroni correction (Rice, supra, p. 384) can also be applied in combination with one of the probability methods for correcting statistical results of one gene versus multiple other genes. In a preferred embodiment, the due-to-chance probability is measured by a Fisher exact test, and the threshold of the due-to-chance probability is set preferably to less than 0.001, more preferably to less than 0.00001.

To determine whether two genes, A and B, have similar co-expression patterns, occurrence data vectors can be generated as illustrated in Table 1. The presence of a gene occurring at least once in a library is indicated by a one, and its absence from the library, by a zero.

TABLE 1
Occurrence Data for Genes A and B

| | Library 1 | Library 2 | Library 3 | . . . | Library N |
|---|---|---|---|---|---|
| Gene A | 1 | 1 | 0 | . . . | 0 |
| Gene B | 1 | 0 | 1 | . . . | 0 |

For a given pair of genes, the occurrence data in Table 1 can be summarized in a 2×2 contingency table.

TABLE 2
Contingency Table for Co-occurrences of Genes A and B

| | Gene A Present | Gene A Absent | Total |
|---|---|---|---|
| Gene B Present | 8 | 2 | 10 |
| Gene B Absent | 2 | 18 | 20 |
| Total | 10 | 20 | 30 |

Table 2 presents co-occurrence data for gene A and gene B in a total of 30 libraries. Both gene A and gene B occur 10 times in the libraries. Table 2 summarizes and presents: 1) the number of times gene A and B are both present in a library; 2) the number of times gene A and B are both absent in a library; 3) the number of times gene A is present, and gene B is absent; and 4) the number of times gene B is present, and gene A is absent. The upper left entry is the number of times the two genes co-occur in a library, and the middle right entry is the number of times neither gene occurs in a library. The off diagonal entries are the number of times one gene occurs, and the other does not. Both A and B are present eight times and absent 18 times. Gene A is present, and gene B is absent, two times; and gene B is present, and gene A is absent, two times. The probability (“p-value”) that the above association occurs due to chance as calculated using a Fisher exact test is 0.0003. Associations are generally considered significant if a p-value is less than 0.01 or 1.0e-2 (Agresti, supra; Rice, supra).

This method of estimating the probability for coexpression of two genes makes several assumptions. The method assumes that the libraries are independent and are identically sampled. However, in practical situations, the selected cDNA libraries are not entirely independent, because more than one library may be obtained from a single subject or tissue. Nor are they entirely identically sampled, because different numbers of cDNAs may be sequenced from each library. The number of cDNAs sequenced typically ranges from 5,000 to 10,000 cDNAs per library. In addition, because a Fisher exact coexpression probability is calculated for each gene versus 37,071 other assembled genes that occur in at least five libraries, a Bonferroni correction for multiple statistical tests is used.

Using the method of the present invention, we have identified cDNAs that exhibit significant association or co-expression probability with known genes that are specific to cancer or disorders associated with cell differentiation. The results presented in Example VI show the direct or indirect associations among expression of novel cDNAs and known retinoic acid induced genes and cell differentiation genes. These genes are RAI1, CYP26, laminin β-2, RBPR-p63, P97, lamin A, β-2-m, APLP2, collagens, notch 3, PDGF, fibulin-2, fibrillin, IGF-I, RPK-RI, SWAP, EMP, YPT3/rab 11, ERK-1, and β4 integrin. Therefore, the five novel cDNAs, SEQ ID NOs1-5 of the Sequence Listing, are useful as surrogate markers for the co-expressed genes in diagnosis, prognosis, or treatment of cancer and disorders associated with cell differentiation. Further, the proteins or peptides expressed from the novel cDNAs are either potential therapeutics or targets for the identification or development of therapeutics.

Therefore, in one embodiment, the present invention encompasses a combination comprising a plurality of cDNAs having the nucleic acid sequences of SEQ ID NOs: 1-5 or the complements thereof. These five cDNAs are shown by the method of the present invention to have significant co-expression with known retinoic acid-induced cell differentiation genes. The invention also provides a cDNA, its complement, and a probe comprising the cDNA selected from SEQ ID NOs: 1-5. Variants typically have at least about 70%, more preferably at least about 85%, and most preferably at least about 95% nucleic acid sequence identity to at least one of these sequences.

The cDNA or the encoded protein may be used to search against the GenBank primate (pri), rodent (rod), mammalian (mam), vertebrate (vrtp), and eukaryote (eukp) databases, SwissProt, BLOCKS (Bairoch et al. (1997) Nucleic Acids Res 25: 217-221), PFAM, and other databases that contain previously identified and annotated motifs, sequences, and gene functions. Methods that search for primary sequence patterns with secondary structure gap penalties (Smith et al. (1992) Protein Engineering 5: 35-51) as well as algorithms such as Basic Local Alignment Search Tool (BLAST; Altschul (1993) J Mol Evol 36: 290-300; Altschul et.al.(1990) J Mol Biol 215: 403-410), BLOCKS (Henikoff and Henikoff (1991) Nucleic Acids Res 19: 6565-6572), Hidden Markov Models (HMM; Eddy (1996) Cur Opin Str Biol 6: 361-365; Sonnhammer et al. (1997) Proteins 28: 405-420), and the like, can be used to manipulate and analyze nucleotide and amino acid sequences. These databases, algorithms and other methods are well known in the art and are described in Ausubel et al. (1997; Short Protocols in Molecular Biology, John Wiley & Sons, New York N.Y., unit 7.7) and in Meyers (1995; Molecular Biology and Biotechnology, Wiley VCH, New York, N.Y., p 856-853).

Also encompassed by the invention are polynucleotides that are capable of hybridizing to SEQ ID NOs: 1-5, and fragments thereof under stringent conditions. Stringent conditions can be defined by salt concentration, temperature, and other chemicals and conditions well known in the art. Conditions can be selected, for example, by varying the concentrations of salt in the prehybridization, hybridization, and wash solutions or by varying the hybridization and wash temperatures. With some substrates, the temperature can be decreased by adding formamide to the prehybridization and hybridization solutions.

Hybridization can be performed at low stringency, with buffers such as 5×SSC (saline sodium citrate) with 1% sodium dodecyl sulfate (SDS) at 60° C., which permits complex formation between two nucleic acid sequences that contain some mismatches. Subsequent washes are performed at higher stringency with buffers such as 0.2×SSC with 0.1% SDS at either 45° C. (medium stringency) or 68° C. (high stringency), to maintain hybridization of only those complexes that contain completely complementary sequences. Background signals can be reduced by the use of detergents such as SDS, sarcosyl, or TRITON X-100 (Sigma-Aldrich, St. Louis, Mo.), and/or a blocking agent, such as salmon sperm DNA. Hybridization methods are described in detail in Ausubel (supra, units 2.8-2.11, 3.18-3.19 and 4-6-4.9) and Sambrook et al. (1989; Molecular Cloning, A Laboratory Manual, Cold Spring Harbor Press, Plainview, N.Y.)

A cDNA can be extended utilizing a partial nucleotide sequence and employing various PCR-based methods known in the art to detect upstream sequences such as promoters and other regulatory elements. (See, e.g., Dieffenbach and Dveksler (1995) PCR Primer, a Laboratory Manual, Cold Spring Harbor Press, Plainview, N.Y.). Additionally, an XL-PCR kit (Applied Biosystems (ABI), Foster City, Calif.), nested primers, and commercially available cDNA libraries (Invitrogen, Carlsbad, Cailf.) or genomic libraries (Clontech, Palo Alto, Cailf.) may be used to extend the sequence. For all PCR-based methods, primers may be designed using commercially available software (LASERGENE software, DNASTAR) or another program, to be about 15 to 30 nucleotides in length, to have a GC content of about 50%, and to form a hybridization complex at temperatures of about 68° C. to 72° C.

In another aspect of the invention, the cDNA can be cloned into a recombinant vector that directs the expression of the protein, or structural or functional portions thereof, in host cells. Due to the inherent degeneracy of the genetic code, other DNA sequences which encode substantially the same or a functionally equivalent amino acid sequence may be produced and used to express the protein encoded by the cDNA. The nucleotide sequences of the present invention can be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

In order to express a biologically active protein, the cDNA or derivatives thereof, may be inserted into an expression vector, i.e., a vector which contains the elements for transcriptional and translational control of the inserted coding sequence in a particular host. These elements include regulatory sequences, such as enhancers, constitutive and inducible promoters, and 5′ and 3′ untranslated regions. Methods which are well known to those skilled in the art may be used to construct such expression vectors. These methods include in vitro recombinant DNA techniques, synthetic techniques, and in vivo genetic recombination (Sambrook, supra; Ausubel, supra).

A variety of expression vector/host cell systems may be utilized to express the cDNA. These include, but are not limited to, microorganisms such as bacteria transformed with recombinant bacteriophage, plasmid, or cosmid expression vectors; yeast transformed with yeast expression vectors; insect cell systems infected with baculovirus vectors; plant cell systems transformed with viral or bacterial expression vectors; or animal cell systems. For long term production of recombinant proteins in mammalian systems, stable expression in cell lines is preferred. For example, the cDNA can be transformed into cell lines using expression vectors which may contain viral origins of replication and/or endogenous expression elements and a selectable or visible marker gene on the same or on a separate vector. The invention is not to be limited by the vector or host cell employed.

In general, host cells that contain the cDNA and that express the protein may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or amino acid sequences. Immunological methods for detecting and measuring the expression of the protein using either specific polyclonal or monoclonal antibodies are known in the art. Examples of such techniques include enzyme-linked immunosorbent assays (ELISAs), radioimmunoassays (RIAs), and fluorescence activated cell sorting (FACS).

Host cells transformed with the cDNA may be cultured under conditions for the expression and recovery of the protein from cell culture. The protein produced by a transgenic cell may be secreted or retained intracellularly depending on the sequence and/or the vector used. As will be understood by those of skill in the art, expression vectors containing the cDNA may be designed to contain signal sequences which direct secretion of the protein through a prokaryotic or eukaryotic cell membrane.

In addition, a host cell strain may be chosen for its ability to modulate expression of the inserted sequences or to process the expressed protein in the desired fashion. Such modifications of the protein include, but are not limited to, acetylation, carboxylation, glycosylation, phosphorylation, lipidation, and acylation. Post-translational processing which cleaves a “prepro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the ATCC (Manassas, Va.) and may be chosen to ensure the correct modification and processing of the expressed protein.

In another embodiment of the invention, natural, modified, or recombinant nucleic acid sequences are ligated to a heterologous sequence resulting in translation of a fusion protein containing heterologous protein moieties in any of the aforementioned host systems. Such heterologous protein moieties facilitate purification of fusion proteins using commercially available affinity matrices. Such moieties include, but are not limited to, glutathione S-transferase, maltose binding protein, thioredoxin, calmodulin binding peptide, 6-His, FLAG, c-myc, hemaglutinin, and monoclonal antibody epitopes.

In another embodiment, the cDNAs, wholly or in part, are synthesized using chemical or enzymatic methods well known in the art (Caruthers et al. (1980) Nucl Acids Symp Ser (7) 215-233; Ausubel, supra). For example, peptide synthesis can be performed using various solid-phase techniques (Roberge et al. (1995) Science 269: 202-204), and machines such as the ABI 431A peptide synthesizer (ABI) can be used to automate synthesis. If desired, the amino acid sequence may be altered during synthesis and/or combined with sequences from other proteins to produce a variant.

Screening, Diagnostics and Therapeutics

The cDNAs can be used as surrogate markers in diagnosis, prognosis, treatment, and selection and evaluation of therapies for cancer and disorders associated with cell differentiation, both as defined herein.

The cDNAs may be used to screen a plurality of molecules for specific binding affinity. The assay can be used to screen a plurality of DNA molecules, RNA molecules, peptide nucleic acids, peptides, ribozymes, antibodies, agonists, antagonists, immunoglobulins, inhibitors, proteins including transcription factors, enhancers, repressors, and drugs and the like which regulate the activity of the polynucleotide in the biological system. The assay involves providing a plurality of molecules, contacting the cDNAs of the combination with the plurality of molecules under conditions suitable to allow specific binding, and detecting specific binding to identify at least one molecule which specifically binds the cDNA

Similarly the proteins or portions thereof may be used to screen libraries of molecules or compounds in any of a variety of screening assays. The portion of a protein employed in such screening may be free in solution, affixed to an abiotic or biotic substrate (e.g. borne on a cell surface), or located intracellularly. Specific binding between the protein and the molecule may be measured. The assay can be used to screen a plurality of DNA molecules, RNA molecules, PNAs, peptides, mimetics, ribozymes, antibodies, agonists, antagonists, immunoglobulins, inhibitors, peptides, polypeptides, drugs and the like, which specifically bind the protein. One method for high throughput screening using very small assay volumes and very small amounts of test compound is described in U.S. Pat. No. 5,876,946, incorporated herein by reference, which screens large numbers of molecules for enzyme inhibition or receptor binding.

In one preferred embodiment, the cDNAs are used for diagnostic purposes to determine the absence, presence, or altered—increased or decreased compared to a normal standard—expression of the gene. The polynucleotide consists of complementary RNA and DNA molecules, branched nucleic acids, and/or PNAs. In one alternative, the polynucleotides are used to detect and quantify gene expression in samples in which expression of the cDNA is correlated with disease. In another alternative, the cDNA can be used to detect genetic polymorphisms associated with a disease. These polymorphisms may be detected in the transcript cDNA.

The specificity of the probe is determined by whether it is made from a unique region, a regulatory region, or from a conserved motif. Both probe specificity and the stringency of diagnostic hybridization or amplification (maximal, high, intermediate, or low) will determine whether the probe identifies only naturally occurring, exactly complementary sequences, allelic variants, or related sequences. Probes designed to detect related sequences should preferably have at least 50% sequence identity to any of the polynucleotides encoding the protein.

Methods for producing hybridization probes include the cloning of nucleic acid sequences into vectors for the production of RNA probes. Such vectors are known in the art, are commercially available, and may be used to synthesize RNA probes in vitro by adding RNA polymerases and labeled nucleotides. Hybridization probes may incorporate nucleotides labeled by a variety of reporter groups including, but not limited to, radionuclides such as ³²P or ³⁵S, enzymatic labels such as alkaline phosphatase coupled to the probe via avidin/biotin coupling systems, fluorescent labels, and the like. The labeled cDNAs may be used in Southern or northern analysis, dot blot, or other membrane-based technologies; in PCR technologies; and in microarrays utilizing samples from subjects to detect altered protein expression.

The cDNA can be labeled by standard methods and added to a sample from a subject under conditions for the formation and detection of hybridization complexes. After incubation the sample is washed, and the signal associated with hybrid complex formation is quantitated and compared with a standard value. Standard values are derived from any control sample, typically one that is free of the suspect disease. If the amount of signal in the subject sample is altered in comparison to the standard value, then the presence of altered levels of expression in the sample indicates the presence of the disease. Qualitative and quantitative methods for comparing the hybridization complexes formed in subject samples with previously established standards are well known in the art.

Such assays may also be used to evaluate the efficacy of a particular therapeutic treatment regimen in animal studies, in clinical trials, or to monitor the treatment of an individual subject. Once the presence of disease is established and a treatment protocol is initiated, hybridization or amplification assays can be repeated on a regular basis to determine if the level of expression in the patient begins to approximate that which is observed in a healthy subject. The results obtained from successive assays may be used to show the efficacy of treatment over a period ranging from several days to many years.

The cDNAs may be used for the diagnosis of a variety of cancers and disorders associated with cell differentiation.

The cDNAs may be used on a substrate such as microarray to monitor the expression patterns. Arrays incorporating cDNAs, proteins, or antibodies may be prepared and analyzed using methods well known in the art. Oligonucleotides or cDNAs may be used as hybridization probes or targets to monitor the expression level of large numbers of genes simultaneously or to identify genetic variants, mutations, and single nucleotide polymorphisms. Proteins may be used to identify ligands, to investigate protein:protein interactions, or to produce a proteomic profile of gene expression (i.e., to detect and quantify expression of a protein in a sample). Antibodies may be also be used produce a proteomic profile of gene expression. Such arrays may be used to determine gene function; to understand the genetic basis of a condition, disease, or disorder; to diagnose a condition, disease, or disorder; and to develop and monitor the activities of therapeutic agents. (See, e.g., Brennan et al. (1995) U.S. Pat. No. 5,474,796; Schena et al. (1996) Proc Natl Acad Sci 93: 10614-10619; Heller et al. (1997) Proc Natl Acad Sci 94: 2150-2155; Heller et al. (1997) U.S. Pat. No. 5,605,662; and deWildt et al. (2000) Nature Biotechnol 18: 989-994.)

In another embodiment, antibodies or Fabs comprising an antigen binding site that specifically binds the protein may be used for the diagnosis of diseases characterized by the over-or-under expression of the protein. A variety of protocols for measuring protein expression, including ELISAs, RIAs, and FACS, are well known in the art and provide a basis for diagnosing altered or abnormal levels of expression. Standard values for protein expression are established by combining samples taken from healthy subjects, preferably human, with antibody to the protein under conditions for complex formation. The amount of complex formation may be quantitated by various methods, preferably by photometric means. Quantities of the protein expressed in disease samples are compared with standard values. Deviation between standard and subject values establishes the parameters for diagnosing or monitoring disease. Alternatively, one may use competitive drug screening assays in which neutralizing antibodies capable of binding specifically with the protein compete with a test compound. Antibodies can be used to detect the presence of any peptide which shares one or more antigenic determinants with the protein. In one aspect, the antibodies of the present invention can be used for treatment or monitoring therapeutic treatment for cancers and disorders associated with cell and tissue development and differentiation.

In another aspect, the cDNA, or its complement, may be used therapeutically for the purpose of expressing mRNA and protein, or conversely to block transcription or translation of the mRNA. Expression vectors may be constructed using elements from retroviruses, adenoviruses, herpes or vaccinia viruses, or bacterial plasmids, and the like. These vectors may be used for delivery of nucleotide sequences to a particular target organ, tissue, or cell population. Methods well known to those skilled in the art can be used to construct vectors to express nucleic acid sequences or their complements. (See, e.g., Maulik et al. (1997) Molecular Biotechnology, Therapeutic Applications and Strategies, Wiley-Liss, New York, N.Y.) Alternatively, the cDNA or its complement, may be used for somatic cell or stem cell gene therapy. Vectors may be introduced in vivo, in vitro, and ex vivo. For ex vivo therapy, vectors are introduced into stem cells taken from the subject, and the resulting transgenic cells are clonally propagated for autologous transplant back into that same subject. Delivery of the cDNA by transfection, liposome injections, or polycationic amino polymers may be achieved using methods which are well known in the art. (See, e.g., Goldman et al. (1997) Nature Biotechnol 15: 462-466.) Additionally, endogenous gene expression may be inactivated using homologous recombination methods which insert an inactive gene sequence into the coding region or other targeted region of the cDNA. (See, e.g. Thomas et al. (1987) Cell 51: 503-512.)

Vectors containing the cDNA can be transformed into a cell or tissue to express a missing protein or to replace a nonfunctional protein. Similarly a vector constructed to express the complement of the cDNA can be transformed into a cell to downregulate the protein expression. Complementary or antisense sequences may consist of an oligonucleotide derived from the transcription initiation site; nucleotides between about positions −10 and +10 from the ATG are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee et al. In: Huber and Carr (1994) Molecular and Immunologic Approaches, Futura Publishing, Mt. Kisco, N.Y., pp. 163-177.)

Ribozymes, enzymatic RNA molecules, may also be used to catalyze the cleavage of mRNA and decrease the levels of particular mRNAs, such as those comprising the cDNAs of the invention. (See, e.g., Rossi (1994) Current Biology 4: 469-471.) Ribozymes may cleave mRNA at specific cleavage sites. Alternatively, ribozymes may cleave mRNAs at locations dictated by flanking regions that form complementary base pairs with the target mRNA. The construction and production of ribozymes is well known in the art and is described in Meyers (supra).

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiester linkages within the backbone of the molecule. Alternatively, nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases, may be included.

Further, an antagonist, or an antibody that binds specifically to the protein may be administered to a subject to treat a cancer or a disorder associated with cell differentiation. The antagonist, antibody, or fragment may be used directly to inhibit the activity of the protein or indirectly to deliver a therapeutic agent to cells or tissues which express the protein. The therapeutic agent may be a cytotoxic agent selected from a group including, but not limited to, abrin, ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin A and 40, radioisotopes, and glucocorticoid.

Antibodies to the protein may be produced using methods that are well known in the art. Such antibodies may include, but are not limited to, polyclonal, monoclonal, chimeric, and single chain antibodies, Fab fragments, and fragments produced by a Fab expression library. Neutralizing antibodies, such as those which inhibit dimer formation, are especially preferred for therapeutic use. Monoclonal antibodies to the protein may be prepared using any technique which provides for the production of antibody molecules by continuous cell lines in culture. These include, but are not limited to, the hybridoma, the human B-cell hybridoma, and the EBV-hybridoma techniques. In addition, techniques developed for the production of chimeric antibodies can be used. (See, e.g., Pound (1998) Immunochemical Protocols, Methods Mol Biol Vol. 80). Alternatively, techniques described for the production of single chain antibodies may be employed. Fabs which contain specific binding sites for the protein may also be generated. Various immunoassays may be used to identify antibodies having the desired specificity. Numerous protocols for competitive binding or immunoradiometric assays using either polyclonal or monoclonal antibodies with established specificities are well known in the art.

Yet further, an agonist of the protein may be administered to a subject to treat or prevent a disease associated with decreased expression, longevity or activity of the protein.

An additional aspect of the invention relates to the administration of a pharmaceutical or sterile composition, in conjunction with a pharmaceutically acceptable carrier, for any of the therapeutic applications discussed above. Such pharmaceutical compositions may consist of the protein or antibodies, mimetics, agonists, antagonists, or inhibitors of the protein. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a subject alone or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Mack Publishing, Easton, Pa.).

For any compound, the therapeutically effective dose can be estimated initially either in cell culture assays or in animal models such as mice, rats, rabbits, dogs, or pigs. An animal model may also be used to determine the concentration range and route of administration. Such information can then be used to determine useful doses and routes for administration in humans.

A therapeutically effective dose refers to that amount of active ingredient which ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the therapeutic compositions described above may be applied to any subject in need of such therapy, including, but not limited to, mammals such as dogs, cats, cows, horses, rabbits, monkeys, and most preferably, humans.

Stem Cells and Their Use

SEQ ID NOs: 1-5 may be useful in the differentiation of stem cells. Eukaryotic stem cells are able to differentiate into the multiple cell types of various tissues and organs and to play roles in embryogenesis and adult tissue regeneration (Gearhart (1998) Science 282: 1061-1062; Watt and Hogan (2000) Science 287: 1427-1430). Depending on their source and developmental stage, stem cells may be totipotent with the potential to create every cell type in an organism and to generate a new organism, pluripotent with the potential to give rise to most cell types and tissues, but not a whole organism; or multipotent cells with the potential to differentiate into a limited number of cell types. Stem cells may be transfected with polynucleotides which may be transiently expressed or may be integrated within the cell as transgenes.

Embryonic stem (ES) cell lines are derived from the inner cell masses of human blastocysts and are pluripotent (Thomson et al. (1998) Science 282: 1145-1147). They have normal karyotypes and express high levels of telomerase which prevents senescence and allows the cells to replicate indefinitely. ES cells produce derivatives that give rise to embryonic epidermal, mesodermal and endodermal cells. Embryonic germ (EG) cell lines, which are produced from primordial germ cells isolated from gonadal ridges and mesenteries, also show stem cell behavior (Shamblott et al. (1998) Proc Natl Acad Sci 95: 13726-13731). EG cells have normal karyotypes and appear to be pluripotent.

Organ-specific adult stem cells differentiate into the cell types of the tissues from which they were isolated. They maintain their original tissues by replacing cells destroyed from disease or injury. Adult stem cells are multipotent and under proper stimulation can be used to generate cell types of various other tissues (Vogel (2000) Science 287: 1418-1419). Hematopoietic stem cells from bone marrow provide not only blood and immune cells, but can also be induced to transdifferentiate to form brain, liver, heart, skeletal muscle and smooth muscle cells. Similarly mesenchymal stem cells can be used to produce bone marrow, cartilage, muscle cells, and some neuron-like cells, and stem cells from muscle have the ability to differentiate into muscle and blood cells (Jackson et al. (1999) Proc Natl Acad Sci 96: 14482-14486). Neural stem cells, which produce neurons and glia, may also be induced to differentiate into heart, muscle, liver, intestine, and blood cells (Kuhn and Svendsen (1999) BioEssays 21: 625-630); Clarke et al. (2000) Science 288: 1660-1663; Gage (2000) Science 287: 1433-1438; and Galli et al. (2000) Nature Neurosci 3: 986-991).

Neural stem cells may be used to treat neurological disorders such as Alzheimer disease, Parkinson disease, and multiple sclerosis and to repair tissue damaged by strokes and spinal cord injuries. Hematopoietic stem cells may be used to restore immune function in immunodeficient patients or to treat autoimmune disorders by replacing autoreactive immune cells with normal cells to treat diseases such as multiple sclerosis, scleroderma, rheumatoid arthritis, and systemic lupus erythematosus. Mesenchymal stem cells may be used to repair tendons or to regenerate cartilage to treat arthritis. Liver stem cells may be used to repair liver damage. Pancreatic stem cells may be used to replace islet cells to treat diabetes. Muscle stem cells may be used to regenerate muscle to treat muscular dystrophies. (See, for example, Fontes and Thomson (1999) BMJ 319: 1-3; Weissman (2000) Science 287: 1442-1446; Marshall (2000) Science 287: 1419-1421; and Marmont (2000) Ann Rev Med 51: 115-134.)

### EXAMPLES

I cDNA Library Construction

Fibroblast Libraries

The FIBRUNT01 cDNA library was constructed using 0.8 μg of polyA RNA isolated from untreated fibroblasts derived from dermal fibroblast tissue removed from the breast of a 31-year-old Caucasian female.

The FIBRTXT02 cDNA library was constructed from 2.5 μg of polyA RNA isolated from fibroblasts from the same donor and which had been treated with 1 μM, 9-cis retinoic acid for 20 hours prior to library construction.

Bone Marrow Libraries

The BMARUNT02 cDNA library was constructed using 1.5 μg of polyA RNA isolated from untreated SH-SY5Y cells derived from a metastatic bone marrow neuroblastoma, removed from a 4-year-old Caucasian female, and maintained in MEM/Ham's F-12 with 10% fetal calf serum.

Cells used to construct the BMARTXT02 cDNA library were cultured in two flasks for 4 days in the presence of 10 μM retinoic acid.

The frozen tissues were homogenized and lysed in TRIZOL reagent (Invitrogen), using a POLYTRON homogenizer (Brinkmann Instruments, Westbury, N.Y.). After a brief incubation on ice, chloroform was added (1:5 v/v), and the lysate was centrifuged. The upper chloroform layer was removed, the aqueous phase containing the RNA was transferred to a fresh tube, the RNA precipitated with isopropanol, resuspended in DEPC-treated water, and treated with DNAse for 25 min at 37° C. If necessary, the RNA was re-extracted before isolation with the OLIGOTEX kit (Qiagen, Chatsworth, Calif.) and used to construct the cDNA library.

Fibroblast mRNA was handled according to the recommended protocols in the SUPERSCRIPT plasmid system (Invitrogen). The cDNAs were fractionated on a SEPHAROSE CL4B column (Amersham Pharmacia Biotech (APB), Piscataway, N.J.), and those cDNAs exceeding 400 bp were ligated into pINCY plasmid (Incyte Genomics, Palo Alto, Calif.) and transformed into DH5α competent cells (Invitrogen).

Bone marrow cDNA synthesis was initiated using a NotI-anchored oligo d(T) primer. Double-stranded cDNA was blunted, ligated to EcoRI adaptors, digested with NotI, size-selected, and cloned into the NotI and EcoRI sites of the pINCY vector (Incyte Genomics) and transformed into competent cells.

II Isolation and Sequencing of cDNA Clones

Plasmid DNA was released from the cells and purified using the REAL PREP 96 plasmid kit (Qiagen). The recommended protocol was employed except for the following changes: 1) the bacteria were cultured in 1 ml of sterile TERRIFIC BROTH (BD Biosciences, Sparks, Md.) with carbenicillin at 25 mg/l and glycerol at 0.4%; 2) the cultures were incubated for 19 hours after the wells were inoculation and then lysed with 0.3 ml of lysis buffer; 3) following isopropanol precipitation, the DNA pellet was resuspended in 0.1 ml of distilled water. After the last step in the protocol, samples were transferred to a 96-well block for storage at 40° C.

The cDNAs were prepared using a MICROLAB 2200 system (Hamilton, Reno, Nev.) in combination with DNA ENGINE thermal cyclers (MJ Research, Watertown, Mass.). The cDNAs were sequenced by the method of Sanger and Coulson (1975; J Mol Biol 94: 441f) using ABI PRISM 377 DNA sequencing systems (ABI). Most of the sequences were sequenced using standard ABI protocols and kits (ABI) at solution volumes of 0.25x-1.0x. In the alternative, some of the sequences were sequenced using solutions and dyes from APB.

III Selection, Assembly, and Characterization of Sequences

The sequences used for co-expression analysis were assembled from EST sequences, 5′ and 3′ long read sequences, and full length coding sequences. Selected assembled sequences were expressed in at least three cDNA libraries.

The assembly process is described as follows. EST sequence chromatograms were processed and verified. Quality scores were obtained using PHRED (Ewing et al. (1998) Genome Res 8: 175-185; Ewing and Green (1998) Genome Res 8: 186-194), and edited sequences were loaded into a relational database management system (RDBMS). The sequences were clustered using BLAST with a product score of 50. All clusters of two or more sequences created a bin which represents one transcribed gene.

Assembly of the component sequences within each bin was performed using a modification of Phrap, a publicly available program for assembling DNA fragments (Green, P. University of Washington, Seattle, Wash.). Bins that showed 82% identity from a local pair-wise alignment between any of the consensus sequences were merged.

Bins were annotated by screening the consensus sequence in each bin against public databases, such as GBpri and GenPept from NCBI. The annotation process involved a FASTn screen against the GBpri database in GenBank. Those hits with a percent identity of greater than or equal to 75% and an alignment length of greater than or equal to 100 base pairs were recorded as homolog hits. The residual unannotated sequences were screened by FASTx against GenPept. Those hits with an E value of less than or equal to 10⁻⁸ were recorded as homolog hits.

Sequences were then reclustered using BLASTn and Cross-Match, a program for rapid amino acid and nucleic acid sequence comparison and database search (Green, supra), sequentially. Any BLAST alignment between a sequence and a consensus sequence with a score greater than 150 was realigned using cross-match. The sequence was added to the bin whose consensus sequence gave the highest Smith-Waterman score (Smith et al. (1992) Protein Engineering 5: 35-51) amongst local alignments with at least 82% identity. Non-matching sequences were moved into new bins, and assembly processes were repeated.

IV Library Subtraction

A profile of the polynucleotides that reflect gene transcription activity in a particular tissue at a particular time is defined as a “transcript image”. Such profiles are produced by sequencing cDNAs and then naming, matching, and counting all copies of related clones and arranging them in order of abundance. The process of producing a comparative transcript image was fully described in U.S. Pat. No. 5,840,484, incorporated herein by reference.

Subtractions between transcript images show the differences occurring between cDNAs produced in untreated and retinoic acid treated fibroblast and bone marrow libraries. The FIBRUNT1 library contained 3781 cDNA clones, and the FIBRTXT02 library, 3849; the BMARUNT02 library contained 3715 cDNA clones, and the BMARTXT02, 3375.

Large numbers of mRNA transcripts, as represented by their respective cDNA clones, were compared using computer-based or “electronic subtraction” methods. For purposes of example, electronic subtraction between any two transcript images parallels hybrid subtraction between any two cDNA libraries using techniques that are known to those of skill in the art (Meyers, supra, pp. 698-699).

V Description of Known Genes

Retinoic Acid Induced Genes

| Name | Description |
|---|---|
| RAI-1/2 | RAI-1 and -2 are retinoic acid-metabolizing cytochromes which regulate retinoic acid |
| | metabolism. RAI-2 is predominantly expressed in the adult cerebellum and is responsible for |
| | all-trans-retinoic acid metabolism. RAI-1 is a novel polyglutamine encoding gene that is |
| | deleted in Smith-Magenis syndrome patients. (White et al. (1996) J Biol Chem 271:29922- |
| | 29927; White et al. (2000) Proc Natl Acad Sci 97:6403-6408; and Loudig et al. (2000) Mol |
| | Endocrinol 14:1483-1497). |
| CYP26 | Retinoic acid hydroxylase (CYP26), is highly specific for all-trans-RA, can be induced |
| | through RA receptors in human breast and colon carcinoma cells, and is a key enzyme in |
| | neuronal differentiation of embryonal carcinoma cells (Marikar et al. (1998) J Invest |
| | Dermatol 111:434-439; Sonneveld et al. (1999) Dev Biol 213:390-404; and Sonneveld et al. |
| | (1998) Cell Growth Differ 9:629-637). |
| B-2 laminin | Beta 2 laminin, is a component of the extracellular matrix that affects retinal development, |
| | is lacking in congenital muscular dystrophy, and has been implicated in cell growth inhibition |
| | and differentiation in testicular carcinoma cells (Bielinska and Wilson (1997) Mech Dev |
| | 65:43-54;Clifford et al. (1999) Cancer Res 59:14-18; and Ueno et al. (1997) Hum Cell |
| | 10:151-158). |
| RBPR-p63 | In one form, retinol-binding-protein receptor protein 63 (RBPR-p63) is an integral membrane |
| | protein of the retinal epithelium where it is part of the receptor-binding complex that |
| | mediates the uptake RBP-bound vitamin A. In another form, it acts with other genes to |
| | transactivate the promoter of keratinocyte differentiation genes (Bavik et al. (1993) J Biol |
| | Chem 268:20540-20546; Johansson et al. (1997) Anat Embryol 195:483-490; De Laurenzi et |
| | al.(2000) Biochem Biophys Res Commun 273:342-346 and Levrero et al. (2000) J Cell Sci |
| | 113:1661-1670). |
| P97 | The tyrosine kinase phosphorylation of the P97 is associated with different cellular activities |
| | and necessary for cell free constitution of the transitional ER. It has been described as a |
| | translational regulator, implicated in the induction of Myc-intron-binding polypeptides, |
| | MIBP1 and RFX1, during retinoic acid-mediated differentiation of haemopoietic cells, and |
| | associated with allergic asthma response (Imataka et al. (1997) EMBO J 16:817-825; Zajac- |
| | Kaye et al. (2000) Biochem J 345:535-541). |
| lamin A | Lamin A is found in an unpolymerized state throughout the nucleoplasm of daughter cell |
| | nuclei in early Gland gradually becomes incorporated into the peripheral lamina during the |
| | first few hours of this stage of the cell cycle. It is-induced in vitro during differentiation of |
| | F9 and P19 embryonal carcinoma cells (Lebel and Raymond (1987) Biochem Biophys Res |
| | Commun 149:417-423; Mattia et al. (1992) Exp Cell Res 203:449-455; and Okumura et al. |
| | (2000) Biochem Biophys Res Commun 269:197-202). |
| β-2-m | Beta-2-microglobulin (β-2-m) is an amyloid protein implicated in the retinoic acid-induced |
| | differentiation of F9, neuroblastoma cells, and myeloma tumors. It is one of the proteins |
| | diagnostic of segmental glomerulosclerosis, contributing to dialysis-related amyloidosis and |
| | specifically removed during hemodialysis (Eriksson et al. (1986) Cancer Res 46:717-722; |
| | Hanada et al. (1993) Cancer Res 53:4978-4986; Lonergan et al. (1993) Mol Cell Biol |
| | 13:6629-6639, and Segars et al. (1993) Mol Cell Biol 13:6157-6169). |
| APLP2 | The amyloid precursor like protein 2 (APLP2), implicated in Alzheimer's disease, exhibits an |
| | intrinsic affinity for the major histocompatibility complex K(d) molecule and strictly |
| | depends upon the presence of β-2-m. APLP2 gene expression is increased in human |
| | neuroblastoma cells in response to retinoic acid (Beckman and Iverfeldt (1997) Neurosci Lett |
| | 221:73-76). |

Genes Involved in Cell Signaling and Differentiation

| Name | Description |
|---|---|
| Collagens | Collagen distribution is altered in the basement membranes of various adenocarcinomas, in |
| | the serum of patients with metastatic breast cancer and collagen vascular diseases and in |
| | intra-alveolar spaces of patients with interstitial lung disease (Maemura et al. (2000) Oncol |
| | Rep 7:1333-1338; Amenta et al. (2000) Hum Pathol 31:359-366; Horie et al. (2000) J |
| | Rheumatol 27:2378-2381; and Yasui et al. (2000) Clin Appl Thromb Hemost 6:202-205). |
| Notch 3 | The Notch signaling genes are pivotal for cell fate decisions at many stages of development |
| | and particularly during formation of the nervous system. Notch 3 lacks specific epidermal |
| | growth factor repeats and is expressed in proliferating neuroepithelium, during pancreatic |
| | development, and in hematopoiesis (Mitsiadis et al. (1998) Dev Biol 204:420-431; Apelqvist |
| | et al. (1999) Nature 400:877-881; and Singh et al. (2000) Exp Hematol 28:527-534). |
| PDGF | Platelet derived growth factor is expressed during embryogenesis, specifically retinoic acid |
| | stimulates immature lung fibroblast growth via a PDGF-mediated autocrine mechanism and |
| | plays a role in the regulation of oligodendrocyte differentiation in the spinal cord (Baron et |
| | al. (2000) Mol Cell Neurosci 15:314-329; Liebeskind et al. (2000) Am J Physiol Lung Cell |
| | Mol Physiol 279:L81-90; Yen and Varvayanis (2000) In Vitro Cell Dev Biol Anim 36:249- |
| | 255; and Noll and Miller (1994) Development 120:649-660). |
| Fibulin-2 | The extracellular matrix protein, fibulin-2, participates in embryogenesis including |
| | atrioventricular valvuloseptal morphogenesis, and is a stromal component of tumors |
| | (Eisenberg and Markwald (1995) Circ Res 77:1-6; Miosge et al. (1996) Histochem J |
| | 28:109-116). |
| Fibrillin | Retinoic acid affects the EGF-R signaling pathway during differentiation of human |
| | endometrial adenocarcinoma cells and during expression of early markers of precardiac |
| | asymmetry (Eisenberg and Markwald (1995) Circ Res 77:1-6; Smith et al. (1997) Dev Biol |
| | 182:162-171). |
| IGF-I | Expression of insulin-like growth factor I (IGF-I) is regulated by retinoic acid in osteoblasts. |
| | (Gabbitas and Canalis (1997) Cell Physiol 172:253-264). |
| PKR RI | cAMP-dependent protein kinase regulatory subunit RI beta participates in retinoic acid |
| | induced differentiation; in fact, retinoylation of the protein is increased in psoriatic human |
| | fibroblasts (Tournier et al. (1996) J Cell Physiol 167:196-203). |
| SWAP | SWAP is a regulatory gene under hormonal control during neuronal differentiation. |
| EMP | EMP is a protein that promotes terminal differentiation of erythroblasts by suppressing |
| | apoptosis (Hanspal et. al (1998) Blood 92:2940-2950). |
| Rab 11 | YPT3/rab 11 is a member of the ras family of signaling molecules. Expression of rab GTP |
| | binding proteins has been implicated in oligodendrocyte differentiation, and altered growth |
| | regulation and loss of response to retinoic acid accompany tumorigenic transformation of |
| | prostatic cells (Drivas et al. (1991) Oncogene 6:3-9; Lai et al. (1994) Genomics 22:610-616; |
| | Peehl et al. (1999) Anticancer Res 19:3857-3864; and Bouverat et al. (2000) J Neurosci Res |
| | 59:446-453). |
| ERK-1 | ERK- 1 is a member of the Ras-extracellular signal-regulated kinase signaling pathway |
| | involved in brain-derived neurotrophic factor-mediated survival and neurogenesis, in the |
| | induction of erythroid differentiation, and various other cellular differentiation processes |
| | (Encinas et al. (1999) J Neurochem 73:1409-1421; Matsuzaki et al. (2000) Oncogene |
| | 19:1500-1508; Nguyen et al. (2000) J Biol Chem 275:19382-19388). |
| μ4 integrin | Retinoic acid advances expression of β4 integrin which in turn affects development of the |
| | embryonic heart (Hierck et al. (1996) Dev Dyn 207:89-103; Laurikainen et al. (1996) Arch |
| | Dermatol Res 288:270-273). |

VI Co-Expression Among the Known Genes and Novel cDNAs

Using the LIFESEQ GOLD database (Incyte Genomics), five cDNAs that showed significant association with known retinoic acid induced genes were identified. Initially, degree of association between known the known retinoic acid induced genes and cDNAS were measured by probability values using a cutoff p-value less than 0.00001. The process was reiterated so that an initial selection of genes were reduced to the final five cDNAs claimed. The following tabular entries show the p-value for the co-expression of any two genes compared with cell differentiation genes. The cDNAs are identified by their SEQ ID NOs, and the known genes, by their abbreviations as shown herein. For each cDNA, the p-value is the probability that the observed co-expression is due to chance, using the Fisher Exact Test.

| Genes co-expressed with SEQ ID NO:1 | p-value |
|---|---|
| Collagen type I | 6.79e−16 |
| Notch3 | 6.71e−15 |
| PDGF | 4.73e−14 |
| Fibulin-2 | 4.85e−12 |
| Fibrillin | 1.79e−11 |
| IGF-I | 1.18e−10 |
| Collagen type XV alpha-1 | 1.69e−10 |

| Genes co-expressed with SEQ ID NO:2 | p-value |
|---|---|
| PKR RI | 3.86e−12 |
| SWAP | 4.76e−12 |

| Genes co-expressed with SEQ ID NO:3 | p-value |
|---|---|
| YPT3/rab 11 | 1.24e−13 |
| ERK-1 | 1.83e−13 |
| β 4 integrin | 3.68e−13 |

| Genes co-expressed with SEQ ID NO:4 | p-value |
|---|---|
| EMP | 4.89e−14 |

Of the five genes, only SEQ ID NO:5 has homology with a known gene, the integrase interactor 1α protein. The cDNA of SEQ ID NO:5 encodes a member of the SWI/SNF chromatin remodeling complex that regulates transcription factor access to regulatory DNA sequences and has been identified as a tumor suppressor for rhabdoid tumors (Suzuki et al. (1997) Diagn Mol Pathol 6: 326-332; Biegel et al. (1999) Cancer Res 59: 74-79; DeCristofaro et al. (1999) Oncogene 18: 7559-7565; Sevenet et al. (1999) Hum Mol Genet 8: 2359-2368; Sevenet et al. (1999) Am J Hum Genet 65: 1342-1348; and Manda et al. (2000) Cancer Lett 153: 57-61). Upregulation of this gene is consistent with the role of retinoic acid as an anti-tumor agent.

VII Homology Searching of cDNA Clones and Their Deduced Proteins

The cDNAs of the Sequence Listing or their deduced amino acid sequences were used to query databases such as GenBank, SwissProt, BLOCKS, and the like. These databases that contain previously identified and annotated sequences or domains were searched using BLAST or BLAST 2 (Altschul et al. supra; Altschul, supra) to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5: 35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin (supra), BLAST matches between a query sequence and a database sequence were evaluated statistically and only reported when they satisfied the threshold of 10⁻²⁵ for nucleotides and 10⁻¹⁴ for peptides. Homology was also evaluated by product score calculated as follows: the % nucleotide or amino acid identity [between the query and reference sequences] in BLAST is multiplied by the % maximum possible BLAST score [based on the lengths of query and reference sequences] and then divided by 100. In comparison with hybridization procedures used in the laboratory, the electronic stringency for an exact match was set at 70, and the conservative lower limit for an exact match was set at approximately 40 (with 1-2% error due to uncalled bases).

The BLAST software suite, freely available sequence comparison algorithms (NCBI, Bethesda, Md.), includes various sequence analysis programs including “blastn” that is used to align nucleic acid molecules and BLAST 2 that is used for direct pairwise comparison of either nucleic or amino acid molecules. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap×drop-off: 50; Expect: 10; Word Size: 11; and Filter: on. Identity or similarity is measured over the entire length of a sequence or some smaller portion thereof. Brenner et al. (1998; Proc Natl Acad Sci 95: 6073-6078, incorporated herein by reference) analyzed the BLAST for its ability to identify structural homologs by sequence identity and found 30% identity is a reliable threshold for sequence alignments of at least 150 residues and 40%, for alignments of at least 70 residues.

The cDNAs of this application were compared with assembled consensus sequences or templates found in the LIFESEQ GOLD database. Component sequences from cDNA, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another and those having local similarity of at least 82% were combined and reassembled. Bins having templates with less than 95% local identity were split. Templates were subjected to analysis by STITCHER/EXON MAPPER algorithms that analyze the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of ≦1×10⁻⁸. The templates were also subjected to frameshift FASTx against GENPEPT, and homolog match was defined as having an E-value of ≦1×10^{31 8}. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis, Mo.; http://pfam.wustl.edu/).

The cDNA was further analyzed using MACDNASIS PRO software (Hitachi Software Engineering), and LASERGENE software (DNASTAR) and queried against public databases such as the GenBank rodent, mammalian, vertebrate, prokaryote, and eukaryote databases, SwissProt, BLOCKS, PRINTS, PFAM, and Prosite.

VIII Hybridization Technologies and Analyses

Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37 C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris-HCl, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning, Acton, Me.) by ultrasound in 0.1% SDS and acetone, etching in 4% hydrofluoric acid (VWR Scientific Products, West Chester, Pa.), coating with 0.05% aminopropyl silane (Sigma-Aldrich) in 95% ethanol, and curing in a 110 C. oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford, Mass.) for 30 min at 60 C.; then the arrays are washed in 0.2% SDS and rinsed in distilled water as before.

Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100 C. for five min, and briefly centrifuging. The denatured cDNA is then added to a REDIPRIME tube (APB), gently mixed until blue color is evenly distributed, and briefly centrifuged. Five μl of [³²P]dCTP is added to the tube, and the contents are incubated at 37 C. for 10 min. The labeling reaction is stopped by adding 5 μl of 0.2M EDTA, and probe is purified from unincorporated nucleotides using a PROBEQUANT G-50 microcolumn (APB). The purified probe is heated to 100 C. for five min, snap cooled for two min on ice, and used in membrane-based hybridizations as described below.

Probe Preparation for Polymer Coated Slide Hybridization

Hybridization probes derived from mRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5×buffer, 1 μl 0.1 M DTT, 3 μl Cy3 or Cy5 labeling mix, 1 μl RNAse inhibitor, 1 μl reverse transcriptase, and 5 μl 1× yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37 C. for two hr. The reaction mixture is then incubated for 20 min at 85 C., and probes are purified using two successive CHROMA SPIN+TE 30 columns (Clontech). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800 xg, and the pellet is resuspended in 12 μl resuspension buffer, heated to 65 C. for five min, and mixed thoroughly. The probe is heated and mixed as before and then stored on ice. Probe is used in high density array-based hybridizations as described below.

Membrane-based Hybridization

Membranes are pre-hybridized in hybridization solution containing 1% Sarkosyl and 1× high phosphate buffer (0.5 M NaCl, 0.1 M Na₂HPO₄, 5 mM EDTA, pH 7) at 55 C. for two hr. The probe, diluted in 15 ml fresh hybridization solution, is then added to the membrane. The membrane is hybridized with the probe at 55 C. for 16 hr. Following hybridization, the membrane is washed for 15 min at 25 C. in 1 mM Tris (pH 8.0), 1% Sarkosyl, and four times for 15 min each at 25 C. in 1 mM Tris (pH 8.0). To detect hybridization complexes, XOMAT-AR film (Eastman Kodak, Rochester, N.Y.) is exposed to the membrane overnight at −70 C., developed, and examined visually.

Polymer Coated Slide-based Hybridization

Probe is heated to 65 C. for five min, centrifuged five min at 9400 rpm in a 5415 C. microcentrifuge (Eppendorf Scientific, Westbury, N.Y.), and then 18 μl are aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60 C. The arrays are washed for 10 min at 45 C. in 1×SSC, 0.1% SDS, and three times for 10 min each at 45 C. in 0.1×SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of-the two labeled probes is hybridized to the array elements, and signals are examined under conditions in which the emissions from the two different labels are individually detectable. Elements on the array that are hybridized to substantially equal numbers of probes derived from both biological samples give a distinct combined fluorescence (Shalon WO95/35505).

Hybridization complexes are detected with a microscope equipped with an INNOVA 70 mixed gas 10 W laser (Coherent, Santa Clara, Calif.) capable of generating spectral lines at 488 nm for excitation of Cy3 and at 632 nm for excitation of Cy5. The excitation laser light is focused on the array using a 20×microscope objective (Nikon, Melville, N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater, N.J.) corresponding to the two fluorophores. Appropriate filters positioned between the array and the photomultiplier tubes are used to filter the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood, Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

IX Expression Analysis

BLAST was used to search for identical or related molecules in the GenBank or LIFESEQ databases (Incyte Genomics). The product score for human and rat sequences was calculated as follows: the BLAST score is multiplied by the % nucleotide identity and the product is divided by (5 times the length of the shorter of the two sequences), such that a 100% alignment over the length of the shorter sequence gives a product score of 100. The product score takes into account both the degree of similarity between two sequences and the length of the sequence match. For example, with a product score of 40, the match will be exact within a 1% to 2% error, and with a product score of at least 70, the match will be exact. Similar or related molecules are usually identified by selecting those which show product scores between 8 and 40.

Electronic northern analysis was performed at a product score of 70 are shown in FIG. 4. All sequences and cDNA libraries in the LIFESEQ database were categorized by system, organ/tissue and cell type. The categories included cardiovascular system, connective tissue, digestive system, embryonic structures, endocrine system, exocrine glands, female and male genitalia, germ cells, hemic/immune system, liver, musculoskeletal system, nervous system, pancreas, respiratory system, sense organs, skin, stomatognathic system, unclassified/mixed, and the urinary tract. For each category, the number of libraries in which the sequence was expressed were counted and shown over the total number of libraries in that category. In a non-normalized library, expression levels of two or more are significant.

X Complementary Molecules

Molecules complementary to the cDNA, from about 5 (PNA) to about 5000 bp (complement of a cDNA insert), are used to detect or inhibit gene expression. These molecules are selected using LASERGENE software (DNASTAR). Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if appropriate elements for inducing vector replication are used in the transformation/expression system.

Stable transformation of appropriate dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the protein.

XI Protein Expression

Expression and purification of the protein are achieved using either a cell expression system or an insect cell expression system. The pUB6/NV5-His vector system (Invitrogen) is used to express protein in. CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6×His) sequence for rapid purification on PROBOND resin (Invitrogen). Transformed cells are selected on media containing blasticidin.

Spodoptera frugiperda (Sf9) insect cells are infected with recombinant Autographica californica nuclear polyhedrosis virus (baculovirus). The polyhedrin gene is replaced with the cDNA by homologous recombination and the polyhedrin promoter drives cDNA transcription. The protein is synthesized as a fusion protein with 6×his which enables purification as described above. Purified protein is used in the following activity and to make antibodies

XII Production of Antibodies

The protein is purified using polyacrylamide gel electrophoresis and used to immunize mice or rabbits. Antibodies are produced using the protocols below. Alternatively, the amino acid sequence of the expressed protein is analyzed using LASERGENE software (DNASTAR) to determine regions of high antigenicity. An antigenic epitope, usually found near the C-terminus or in a hydrophilic region is selected, synthesized, and used to raise antibodies. Typically, epitopes of about 15 residues in length are produced using an ABI 431A peptide synthesizer (ABI) using FMOC-chemistry and coupled to KLH (Sigma-Aldrich) by reaction with N-maleimidobenzoyl-N-hydroxysuccinimide ester to increase antigenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

XIII Purification of Naturally Occurring Protein Using Specific Antibodies

Naturally occurring or recombinant protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbence of the protein. After coupling, the protein is eluted from the column using a buffer of pH 2-3 or a high concentration of urea or thiocyanate ion to disrupt antibody/protein binding, and the protein is collected.

XIV Screening Molecules for Specific Binding with the cDNA or Protein

The cDNA, or fragments thereof, or the protein, or portions thereof, are labeled with ³²P-dCTP, Cy3-dCTP, or Cy5-dCTP (APB), or with BIODIPY or FITC (Molecular Probes, Eugene, Oreg.), respectively. Libraries of candidate molecules or compounds previously arranged on a substrate are incubated in the presence of labeled cDNA or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XV Two-Hybrid Screen

A yeast two-hybrid system, MATCHMAKER LexA Two-Hybrid system (Clontech Laboratories), is used to screen for peptides that bind the protein of the invention. A cDNA encoding the protein is inserted into the multiple cloning site of a pLexA vector, ligated, and transformed into E. coli. cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubate at 30 C. until the colonies have grown up and are counted. The colonies are pooled in a minimal volume of 1×TE (pH 7.5), replated on SD/-His/-Leu/-Trp/-Ura media supplemented with 2% galactose (Gal), 1% raffinose (Raf), and 80 mg/ml 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (-Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30 C. A sample of the culture is plated on SD/-Trp/-Ura media and incubated at 30 C. until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gal/-Trp/-Ura, and white colonies are isolated and propagated. The pB42AD-cDNA plasmid, which contains a cDNA encoding a protein that physically interacts with the protein, is isolated from the yeast cells and characterized.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

What is claimed is:

1. A combination comprising a plurality of cDNAs that are induced with retinoic acid wherein the cDNAs have the nucleic acid sequences of SEQ ID NOs: 1-5 and complements of nucleic acid sequences of SEQ ID NOs: 1-5.

2. An isolated cDNA comprising a nucleic acid sequence selected from SEQ ID NOs: 1-5 or the complement thereof.

3. A composition comprising the cDNA of claim 2 and a labeling moiety.

4. A method of using a combination to screen a plurality of molecules and compounds to identify at least one molecule or compound which specifically binds a cDNA of the combination, the method comprising:
a) combining the combination of claim 1 with a plurality of molecules and compounds under conditions to allow specific binding; and
b) detecting specific binding, thereby identifying a molecule or compound which specifically binds a cDNA of the combination.

5. A method of using a combination to detect the presence of complementary nucleic acids in a sample comprising:
a) hybridizing the combination of claim 1 with the nucleic acids under conditions to allow formation of one or more hybridization complexes;
b) detecting complex formation; wherein complex formation indicates the presence of complementary nucleic acids in the sample.

6. The method of claim 5 wherein the nucleic acids are amplified prior to hybridization.

7. The method of claim 5 wherein the sample is from a patient with cancer or a disorder associated with cell differentiation.

8. The method of claim 5 wherein the cDNAs of the combination are attached to a substrate.

9. An expression vector comprising a cDNA selected from SEQ ID NOs: 1-4.

10. A host cell comprising the expression vector of claim 9.

11. A method for using a host cell to produce a protein, the method comprising:
a) culturing the host cell of claim 10 under conditions for expression of the protein; and
b) recovering the protein from cell culture.

12. A purified protein or a portion thereof obtained using the method of claim 11.

13. A composition comprising the protein of claim 12 and a pharmaceutical carrier.

14. A method for using a protein to screen a plurality of molecules to identify at least one ligand which specifically binds the protein, the method comprising:
a) combining the protein of claim 12 with the plurality of molecules under conditions to allow specific binding; and
b) detecting specific binding between the protein and ligand, thereby identifying a ligand which specifically binds the polypeptide.

15. A method of using a protein to prepare and purify an antibody comprising:
a) immunizing a animal with a protein of claim 12 under conditions to elicit an antibody response;
b) isolating animal antibodies;
c) attaching the protein to a substrate;
d) contacting the substrate with isolated antibodies under conditions to allow specific binding to the protein;
e) dissociating the antibodies from the protein, thereby obtaining purified antibodies.

16. An isolated antibody which specifically binds a protein of claim 12.

17. A composition comprising an antibody of claim 16 and a labeling moiety.

18. A method for using an antibody to detect expression in a sample, the method comprising:
a) combining the antibody of claim 16 with a sample under conditions which allow the formation of antibody:protein complexes; and
b) detecting complex formation, wherein complex formation indicates expression of the protein in the sample.

19. The method of claim 18 wherein complex formation is compared with standards and is diagnostic of a disorder associated with steroid-responsive tissues or pregnancy.

20. A method for using an antibody to immunopurify a protein comprising:
a) attaching the antibody of claim 16 to a substrate;
b) contacting the antibody with solution containing the protein, thereby forming an antibody:protein complex;
c) dissociating the antibody:protein complex; and
d) collecting the purified protein.
